SEQUENCE LISTING (24 sequences)

SEQ ID NO: 1 - protein, 5 aa, synthetic construct
  VARIANT 5: note = S or G
NYAMX

SEQ ID NO: 2 - protein, 17 aa, synthetic construct
AISWSGGSTSYADSVKG

SEQ ID NO: 3 - protein, 21 aa, synthetic construct
  VARIANT 7: note = F or S
QFSGADXGFGRLGIRGYEYDY

SEQ ID NO: 4 - protein, 130 aa, synthetic construct
  VARIANT 35: note = S or G
  VARIANT 105: note = F or S
EVQLLESGGGSVQPGGSLRLSCAASGRPFSNYAMXWFRQAPGKEREFVSAISWSGGSTSYADSVKGRFTISRDNSKNTLYLQMNSLRAEDTAVYYCAAQFSGADXGFGRLGIRGYEYDYWGQGTQVTVSS

SEQ ID NO: 5 - protein, 5 aa, synthetic construct
SYGMG

SEQ ID NO: 6 - protein, 17 aa, synthetic construct
GISWRGDSTGYADSVKG

SEQ ID NO: 7 - protein, 15 aa, synthetic construct
AAGSAWYGTLYEYDY

SEQ ID NO: 8 - protein, 124 aa, synthetic construct
EVQLVESGGGSVQPGGSLRLSCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTLVTVSS

SEQ ID NO: 9 - protein, 5 aa, synthetic construct
GGGGS

SEQ ID NO: 10 - protein, 14 aa, synthetic construct
AAASDKTHTCPPCP

SEQ ID NO: 11 - protein, 231 aa, synthetic construct
  VARIANT 231: note = K or is absent
AAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 12 - protein, 231 aa, synthetic construct
  VARIANT 231: note = K or is absent
AAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 13 - protein, 130 aa, synthetic construct
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 14 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS

SEQ ID NO: 15 - protein, 21 aa, synthetic construct
QFSGADYGFGRLGIRGYEYDY

SEQ ID NO: 16 - protein, 355 aa, synthetic construct
  VARIANT 355: note = K or is absent
EVQLVESGGGSVQPGGSLRLSCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTLVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 17 - protein, 361 aa, synthetic construct
  VARIANT 361: note = K or is absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGRLGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 18 - protein, 361 aa, synthetic construct
  VARIANT 361: note = K or is absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGRLGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 19 - protein, 21 aa, synthetic construct
QFSGADFGFGRLGIRGYEYDY

SEQ ID NO: 20 - protein, 21 aa, synthetic construct
QFSGADSGFGRLGIRGYEYDY

SEQ ID NO: 21 - protein, 5 aa, synthetic construct
NYAMS

SEQ ID NO: 22 - protein, 5 aa, synthetic construct
NYAMG

SEQ ID NO: 23 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGRLGIRGYEYDYWGQGTQVTVSS

SEQ ID NO: 24 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGRLGIRGYEYDYWGQGTQVTVSS

(19) United States
(12) Patent Application Publication
(10) Pub. No.: US 2026/0152560 A1
(43) Pub. Date: Jun. 4, 2026

(54) ANTIBODIES THAT BIND GAMMA-DELTA T CELL RECEPTORS FOR THE TREATMENT OF CANCER

(71) Applicants: LAVA THERAPEUTICS N.V., Utrecht (NL); SEAGEN INC., Bothell, WA (US)

(72) Inventors: Johannes Jelle VAN DER VLIET, Utrecht (NL); Corinna PALANCA-WESSELS, Bothell, WA (US)

(21) Appl. No.: 19/461,262

(22) Filed: Jan. 27, 2026

Related U.S. Application Data
(63) Continuation of application No. PCT/US2024/039779, filed on Jul. 26, 2024.
(60) Provisional application No. 63/529,252, filed on Jul. 27, 2023.

(51) Int. Cl. C07K 16/28; A61K 39/00; A61K 47/18; A61K 47/26

(52) U.S. Cl. CPC C07K 16/2809; A61K 47/183; A61K 47/26; C07K 16/2863; A61K 2039/54; C07K 2317/31; C07K 2317/565; C07K 2317/569

(57) ABSTRACT

In some embodiments, the present disclosure provides methods of treating a cancer in a subject in need thereof, wherein the cancer is selected from pancreatic cancer, non-small cell lung cancer (NSCLC), colorectal cancer (CRC), and head and neck squamous cell cancer (HNSCC) comprising administering to the subject a bispecific antibody comprising a first antigen-binding region that binds a Vγ9Vδ2 T cell receptor (TCR) a second antigen-binding region that binds to EFGR. In some embodiments, the pancreatic cancer is pancreatic ductal adenocarcinoma.

### CROSS-REFERENCE TO RELATED APPLICATION

This application is a continuation of International PCT Application No. PCT/US2024/039779, filed Jul. 26, 2024, which claims priority to and benefit of U.S. Provisional Application No. 63/529,252, filed Jul. 27, 2023, the entire disclosure of which are incorporated by reference herein in their entirety.

### REFERENCE TO AN ELECTRONIC SEQUENCE LISTING

The contents of the electronic sequence listing (LVAT_029_01US_SeqList_ST26.xml; Size: 27,445 bytes; and Date of Creation: Jan. 8, 2026) are herein incorporated by reference in their entirety.

### BACKGROUND

Gamma-delta (γδ) T cells are T cells that express a T cell receptor (TCR) that is made up of a gamma chain and a delta chain. The majority of γδ T cells express TCRs comprising Vγ9 and Vδ2 regions. Vγ9Vδ2 T cells can react against a wide array of pathogens and tumor cells. This broad reactivity is understood to be conferred by phosphoantigens that are able to specifically activate this T-cell subset in a TCR dependent fashion. The broad antimicrobial and anti-tumor reactivity of Vγ9Vδ2 T-cells suggest a direct involvement in immune control of cancers and infections.

### SUMMARY

The present disclosure provides methods of treating solid tumors, including non-small cell lung cancer, head and neck squamous cell cancer, colorectal cancer, and pancreatic cancer, in a subject in need thereof comprising administration of bispecific antibodies comprising a first antigen binding domain that binds to a Vγ9Vδ2 TCR and a second antigen binding domain that binds to EGFR.

In some embodiments, the present disclosure provides a method of treating pancreatic cancer in a subject in need thereof comprising administering to the subject a bispecific antibody comprising (a) a first antigen-binding region that binds a Vy9Vδ2 T cell receptor (TCR) and comprises (i) a CDR1 of SEQ ID NO: 21, a CDR2 of SEQ ID NO: 2, and a CDR3 of SEQ ID NO: 19, or (ii) a CDR1 of SEQ ID NO: 21, a CDR2 of SEQ ID NO: 2, and a CDR3 of SEQ ID NO: 20; and (b) a second antigen-binding region that binds to EFGR and comprises a CDR1 of SEQ ID NO: 5, a CDR2 of SEQ ID NO: 6, and a CDR3 of SEQ ID NO: 7.

In some embodiments, the present disclosure provides a method of treating pancreatic cancer in a subject in need thereof comprising administering to the subject a bispecific antibody comprising (a) a first antigen-binding domain that binds a Vγ9Vδ2 T cell receptor (TCR) and comprises an amino acid sequence selected from SEQ ID NO:24 and 23 and (b) a second antigen-binding domain that binds to EFGR and comprises an amino acid sequence of SEQ ID NO:8. In some embodiments, the pancreatic cancer is metastatic pancreatic cancer. In some embodiments, the pancreatic cancer is unresectable. In some embodiments, the pancreatic cancer is pancreatic ductal adenocarcinoma. In some embodiments, the pancreatic cancer is relapsed or refractory pancreatic cancer. In some embodiments, the subject has received prior treatment with one or more chemotherapeutic agents and/or one or more immune checkpoint inhibitors. In some embodiments, the one or more chemotherapeutic agents are selected from (a) gemcitabine; and (b) leucovorin calcium (folinic acid), fluorouracil, irinotecan hydrochloride, and oxaliplatin.

In some embodiments, the present disclosure provides a method of treating non-small cell lung cancer (NSCLC) in a subject in need thereof comprising administering to the subject a bispecific antibody comprising (a) a first antigen-binding domain that binds a Vγ9Vδ2 T cell receptor (TCR) and comprises an amino acid sequence selected from SEQ ID NO:24 and 23 and (b) a second antigen-binding domain that binds to EFGR and comprises an amino acid sequence of SEQ ID NO:8. In some embodiments, the NSCLC is relapsed or refractory NSCLC. In some embodiments, the NSCLC is metastatic NSCLC. In some embodiments, the NSCLC is unresectable. In some embodiments, the subject has received prior treatment with one or more chemotherapeutic agents and/or one or more immune checkpoint inhibitors. In some embodiments, the one or more chemotherapeutic agents are platinum-based chemotherapeutic agents and/or the one or more immune checkpoint inhibitors is a PD1 or PDL1 inhibitor.

In some embodiments, the present disclosure provides a method of treating colorectal cancer (CRC) in a subject in need thereof comprising administering to the subject a bispecific antibody comprising (a) a first antigen-binding domain that binds a Vγ9Vδ2 T cell receptor (TCR) and comprises an amino acid sequence selected from SEQ ID NO:24 and 23 and (b) a second antigen-binding domain that binds to EFGR and comprises an amino acid sequence of SEQ ID NO:8. In some embodiments, the CRC is relapsed or refractory CRC. In some embodiments, the CRC is metastatic CRC. In some embodiments, the CRC is unresectable. In some embodiments, the subject has received prior treatment with one or more chemotherapeutic agents and/or one or more immune checkpoint inhibitors. In some embodiments, the one or more chemotherapeutic agents is selected from a fluoropyrimidine, a platinum-based chemotherapeutic, and a topoisomerase inhibitor. In some embodiments, the platinum-based chemotherapeutic is oxaliplatin; and/or the topoisomerase inhibitor is irinotecan or camptothecin. In some embodiments, the immune checkpoint inhibitor is selected from an anti-PD1 or anti-PDL1 antibody.

In some embodiments, the present disclosure provides a method of treating head and neck squamous cell cancer (HNSCC) in a subject in need thereof comprising administering to the subject a bispecific antibody comprising (a) a first antigen-binding domain that binds a Vγ9Vδ2 T cell receptor (TCR) and comprises an amino acid sequence selected from SEQ ID NO: 24 and 23 and (b) a second antigen-binding domain that binds to EFGR and comprises an amino acid sequence of SEQ ID NO:8. In some embodiments, the HNSCC is relapsed or refractory HNSCC. In some embodiments, the HNSCC is metastatic HNSCC. In some embodiments, the HNSCC is unresectable. In some embodiments, the subject has received prior treatment with one or more chemotherapeutic agents and/or one or more immune checkpoint inhibitors. In some embodiments, the one or more chemotherapeutic agents are platinum-based chemotherapeutic agents and/or the one or more immune checkpoint inhibitors is a PD1 or PDL1 inhibitor.

In some embodiments, the subject has not been diagnosed with another malignancy less than 3 years prior to administration of a first dose of the bispecific antibody. In some embodiments, the subject does not have an active central nervous system metastases or leptomeingeal disease. In some embodiments, the subject has not received treatment with an aminobisphosphonate IV less than 4 weeks prior to administration of a first dose of the bispecific antibody. In some embodiments, the subject has not had a thromboembolic phenomenon less than 6 months prior to administration of a first dose of the bispecific antibody.

In some embodiments, the bispecific antibody is formulated in a pharmaceutical composition comprising: 1 mg/mL or 10 mg/ml of the bispecific antibody; 10 mM Histidine; 280 mM Sucrose; 0.02% Polysorbate 80; pH 6.0; and 1 mM Methionine.

In some embodiments, the subject is a human. In some embodiments, the bispecific antibody or pharmaceutical composition comprising the same is administered intravenously.

### DETAILED DESCRIPTION

### Definitions

The term “human Vδ2”, when used herein, refers to the rearranged 02 chain of the Vγ9Vδ2-T cell receptor (TCR). UniProtKB-A0JD36 (A0JD36_HUMAN) gives an example of a variable TRDV2 sequence. Vδ2 is part of the delta chain of the Vγ9Vδ2-TCR. An antibody capable of binding to human Vδ2 may bind an epitope that is entirely located within the variable region or bind an epitope that is located within the constant region or bind an epitope that is a combination of residues of the variable and constant regions of the delta chain.

The term “human Vγ9”, when used herein, refers to the rearranged γ9 chain of the Vγ9Vδ2-T cell receptor (TCR). UniProtKB-Q99603_HUMAN gives an example of a variable TRGV9 sequence.

The term “EGFR”, when used herein, refers to the human EGFR protein (UniProtKB—P00533 (EGFR_HUMAN)).

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The antigen-binding region (or antigen-binding domain) which interacts with an antigen may comprise variable regions of both the heavy and light chains of the immunoglobulin molecule or may be a single-domain antigen-binding region, e.g. a heavy chain variable region only. The constant regions of an antibody, if present, may mediate the binding of the immunoglobulin to host tissues or factors, including various cells of the immune system (such as effector cells and T cells) and components of the complement system such as C1q, the first component in the classical pathway of complement activation.

The Fc region of an immunoglobulin is defined as the fragment of an antibody which would be typically generated after digestion of an antibody with papain which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system.

The term “hinge region” as used herein is intended to refer to the hinge region of an immunoglobulin heavy chain. Thus, for example, the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

Reference to amino acid positions in the Fc region/Fc domain in the present invention is according to the EU-numbering (Edelman et al., Proc Natl Acad Sci USA. 1969 May; 63 (1): 78-85; Kabat et al., Sequences of proteins of immunological interest. 5th Edition-1991 NIH Publication No. 91-3242).

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′)2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise indicated by context. Although such fragments are generally included within the meaning of antibody, they collectively and each independently are unique features of the present invention, exhibiting different biological properties and utility. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies of the invention, the first antigen-binding region or the second antigen-binding region, or both, is a single domain antibody. Single domain antibodies are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single domain antibodies comprise a single CDR1, a single CDR2 and a single CDR3. Examples of single domain antibodies are variable fragments of heavy-chain-only antibodies, antibodies that naturally do not comprise light chains, single domain antibodies derived from conventional antibodies, and engineered antibodies. Single domain antibodies may be derived from any species including mouse, human, camel, llama, shark, goat, rabbit, and cow. For example, single domain antibodies can be derived from antibodies raised in Camelidae species, for example in camel, dromedary, llama, alpaca and guanaco. Like a whole antibody, a single domain antibody is able to bind selectively to a specific antigen. Single domain antibodies may contain only the variable domain of an immunoglobulin chain, i.e. CDR1, CDR2 and CDR3 and framework regions. Such antibodies are also called Nanobody®, or VHH.

The term “immunoglobulin” as used herein is intended to refer to a class of structurally related glycoproteins consisting of two pairs of polypeptide chains, one pair of light (L) chains and one pair of heavy (H) chains, all four potentially inter-connected by disulfide bonds. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. IgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Chothia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “isotype” as used herein, refers to the immunoglobulin (sub) class (for instance IgG1, IgG2, IgG3, IgG4, IgD, IgA, IgE, or IgM) or any allotype thereof, such as IgG1m (za) and IgG1m (f) that is encoded by heavy chain constant region genes. Each heavy chain isotype can be combined with either a kappa (κ) or lambda (A) light chain. An antibody of the invention can possess any isotype.

The term “parent antibody”, is to be understood as an antibody which is identical to an antibody according to the invention, but wherein the parent antibody does not have one or more of the specified mutations. A “variant” or “antibody variant” or a “variant of a parent antibody” of the present invention is an antibody molecule which comprises one or more mutations as compared to a “parent antibody”. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), |
| Residues | and Gln (Q) |
| Aliphatic Uncharged | Gly (G), Ala (A), Val (V), |
| Residues | Leu (L), and Ile (I) |
| Non-polar Uncharged | Cys (C), Met (M), and Pro (P) |
| Residues | |
| Aromatic Residues | Phe (F), Tyr (Y), and Trp (W) |

Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications
of Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, |
| | T, V, W, and Y |
| Negatively charged residues | D and E |
| Polar residues | C, D, E, H, K, N, Q, R, S, |
| | and T |
| Positively charged residues | H, K, and R |
| Small residues | A, C, D, G, N, P, S, T, and V |
| Very small residues | A, G, and S |
| Residues involved in | A, C, D, E, G, H, K, N, Q, |
| turn formation | R, S, P, and T |
| Flexible residues | Q, T, K, S, G, N, D, E, and R |

In the context of the present invention, a substitution in a variant is indicated as: Original amino acid—position—substituted amino acid. The three-letter code, or one letter code, are used, including the codes Xaa and X to indicate amino acid residue. Accordingly, the notation “T366W” means that the variant comprises a substitution of threonine with tryptophan in the variant amino acid position corresponding to the amino acid in position 366 in the parent antibody.

Furthermore, the term “a substitution” embraces a substitution into any one of the other nineteen natural amino acids, or into other amino acids, such as non-natural amino acids. For example, a substitution of amino acid T in position 366 includes each of the following substitutions: 366A, 366C, 366D, 366G, 366H, 366F, 366I, 366K, 366L, 366M, 366N, 366P, 366Q, 366R, 366S, 366E, 366V, 366W, and 366Y.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes. Such epitopes may be on the same or on different target antigens. If the epitopes are on different targets, such targets may be on the same cell or different cells or cell types. In some embodiments, a multispecific antibody may comprise one or more single-domain antibodies.

The term “bispecific antibody” refers to an antibody having specificities for two different, typically non-overlapping, epitopes. Such epitopes may be on the same or different targets. If the epitopes are on different targets, such targets may be on the same cell or different cells or cell types. In some embodiments, a bispecific antibody may comprise one or two single-domain antibodies.

Examples of different classes of multispecific, such as bispecific, antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287 (52): 43331-9, doi: 10.1074/jbc.M112.397869. Epub 2012 Nov. 1), DIG-body and PIG-body (Pharmabcine, WO2010134666, WO2014081202), the Strand Exchange Engineered Domain body (SEEDbody) (EMD Serono), the Biclonics (Merus, WO2013157953), FcΔAdp (Regeneron), bispecific IgG1 and IgG2 (Pfizer/Rinat), Azymetric scaffold (Zymeworks/Merck), mAb-Fv (Xencor), bivalent bispecific antibodies (Roche, WO2009080254) and DuoBody® molecules (Genmab).

Examples of recombinant IgG-like dual targeting molecules include but are not limited to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-April; 5 (2): 208-18), approaches with common light chain, κλBodies (NovImmune, WO2012023053) and CovX-Body® (CovX/Pfizer, Doppalapudi, V. R., et al 2007. Bioorg. Med. Chem. Lett. 17,501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February; 32 (2): 191-8), Ts2Ab (MedImmune/AZ, Dimasi et al. J Mol Biol. 2009 Oct. 30; 393 (3): 672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to ScFv/Fc Fusions (Academic Institution, Pearce et al Biochem Mol Biol Int. 1997 September; 42 (6): 1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual (ScFv) 2-Fab (National Research Center for Antibody Medicine—China).

Examples of Fab fusion bispecific antibodies include but are not limited to F (ab) 2 (Medarex/AMGEN), Dual-Action or Bis-Fab (Genentech), Dock-and-Lock® (DNL) (ImmunoMedics), Bivalent Bispecific (Biotecnoly and Fab-Fv (UCB-Celltech).

Examples of ScFv-, diabody-based and domain antibodies include but are not limited to Bispecific T Cell Engager (BITE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3; 425 (3): 479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88 (6): 667-75), dual targeting Nanobodies® (Ablynx, Hmila et al., FASEB J. 2010), dual targeting heavy chain only domain antibodies.

In the context of antibody binding to an antigen, the terms “binds” or “specifically binds” refer to the binding of an antibody to a predetermined antigen or target (e.g. human V□2 or human EGFR) to which binding typically is with an apparent affinity corresponding to a KD of about 10-6 M or less, e.g. 10-7 M or less, such as about 10⁻⁸ M or less, such as about 10-9 M or less, about 10-10 M or less, or about 10-11 M or even less, e.g. when determined using flow cytometry as described in the Examples herein. Alternatively, KD values can be determined using for instance surface plasmon resonance (SPR) technology in a BIAcore T200 or bio-layer interferometry (BLI) in an Octet RED96 instrument using the antigen as the ligand and the binding moiety or binding molecule as the analyte. Specific binding means that the antibody binds to the predetermined antigen with an affinity corresponding to a KD that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The degree with which the affinity is lower is dependent on the KD of the binding moiety or binding molecule, so that when the KD of the binding moiety or binding molecule is very low (that is, the binding moiety or binding molecule is highly specific), then the degree with which the affinity for the antigen is lower than the affinity for a non-specific antigen may be at least 10,000-fold. The term “KD” (M), as used herein, refers to the dissociation equilibrium constant of a particular interaction between the antigen and the binding moiety or binding molecule.

In the context of the present invention, “competition” or “able to compete” or “competes” refers to any detectably significant reduction in the propensity for a particular binding molecule (e.g. an EGFR antibody) to bind a particular binding partner (e.g. EGFR) in the presence of another molecule (e.g. a different EGFR antibody) that binds the binding partner. Typically, competition means an at least about 25 percent reduction, such as an at least about 50 percent, e.g. an at least about 75 percent, such as an at least 90 percent reduction in binding, caused by the presence of another molecule, such as an antibody, as determined by, e.g., ELISA analysis or flow cytometry using sufficient amounts of the two or more competing molecules, e.g. antibodies. Additional methods for determining binding specificity by competitive inhibition may be found in for instance Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley InterScience N. Y., (1992, 1993), and Muller, Meth. Enzymol. 92, 589-601 (1983)).

In one embodiment, the antibody of the present invention binds to the same epitope on EGFR as antibody 7D12 and/or to the same epitope on Vo2 as antibody 5C8. There are several methods available for mapping antibody epitopes on target antigens known in the art, including but not limited to: crosslinking coupled mass spectrometry, allowing identification of peptides that are part of the epitope, and X-ray crystallography identifying individual residues on the antigen that form the epitope. Epitope residues can be determined as being all amino acid residues with at least one atom less than or equal to 5 Å from the antibody. 5 Å was chosen as the epitope cutoff distance to allow for atoms within a van der Waals radius plus a possible water-mediated hydrogen bond. Next, epitope residues can be determined as being all amino acid residues with at least one atom less than or equal to 8 Å. Less than or equal to 8 Å is chosen as the epitope cutoff distance to allow for the length of an extended arginine amino acid. Crosslinking coupled mass spectrometry begins by binding the antibody and the antigen with a mass labeled chemical crosslinker. Next the presence of the complex is confirmed using high mass MALDI detection. Because after crosslinking chemistry the Ab/Ag complex is extremely stable, many various enzymes and digestion conditions can be applied to the complex to provide many different overlapping peptides. Identification of these peptides is performed using high resolution mass spectrometry and MS/MS techniques. Identification of the crosslinked peptides is determined using mass tag linked to the crosslinking reagents. After MS/MS fragmentation and data analysis, peptides that are crosslinked and are derived from the antigen are part of the epitope, while peptides derived from the antibody are part of the paratope. All residues between the most N- and C-terminal crosslinked residue from the individual crosslinked peptides found are considered to be part of the epitope or paratope. The epitope of antibody 7D12 has been determined by X-ray crystallography, described in Schmitz et al. (2013) Structure 21:1214 and consists of a flat surface on domain III (residues R353, D355, F357, Q384, N420) that corresponds to the domain Ill ligand-binding site.

The terms “first” and “second” antigen-binding regions when used herein do not refer to their orientation/position in the antibody, i.e. they have no meaning with regard to the N- or C-terminus. The terms “first” and “second” only serve to correctly and consistently refer to the two different antigen-binding regions in the claims and the description.

“% sequence identity”, when used herein, refers to the number of identical nucleotide or amino acid positions shared by different sequences (i.e., % identity=# of identical positions/total # of positions×100), taking into account the number of gaps, and the length of each gap, which need to be introduced for optimal alignment. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4.

### Methods of Treatment

In some embodiments, the present disclosure provides a method of treating a cancer in a subject in need thereof, wherein the cancer is selected from pancreatic cancer, non-small cell lung cancer (NSCLC), colorectal cancer (CRC), and head and neck squamous cell cancer (HNSCC) comprising administering to the subject a bispecific antibody comprising (a) a first antigen-binding region that binds a Vγ9Vδ2 T cell receptor (TCR) and comprises (i) a CDR1 of SEQ ID NO: 21, a CDR2 of SEQ ID NO: 2, and a CDR3 of SEQ ID NO: 19, or (ii) a CDR1 of SEQ ID NO: 21, a CDR2 of SEQ ID NO: 2, and a CDR3 of SEQ ID NO: 20; and (b) a second antigen-binding region that binds to EFGR and comprises a CDR1 of SEQ ID NO: 5, a CDR2 of SEQ ID NO: 6, and a CDR3 of SEQ ID NO: 7. In some embodiments, the present disclosure provides methods of treating a cancer in a subject in need thereof, wherein the cancer is selected from pancreatic cancer, non-small cell lung cancer (NSCLC), colorectal cancer (CRC), and head and neck squamous cell cancer (HNSCC) comprising administering to the subject a bispecific antibody comprising (a) a first antigen-binding region that binds a Vγ9Vδ2 T cell receptor (TCR) and comprises an amino acid sequence that is at least 90%, at least 95%, or at least 99% identical to a sequence selected from SEQ ID NO:24 and 23 and (b) a second antigen-binding region that binds to EFGR and comprises an amino acid sequence that is at least 90%, at least 95%, or at least 99% identical to SEQ ID NO:8. In some embodiments, the first antigen binding region comprises or consists of SEQ ID NO:24 or 23 and the second antigen binding region comprises or consists of SEQ ID NO:8. In some embodiments, the pancreatic cancer is pancreatic ductal adenocarcinoma.

In some embodiments, the bispecific antibody comprises a half-life extension domain. In some embodiments, the bispecific antibody has a terminal half-life that is longer than about 168 or longer than about 336 hours when administered to a human subject. The “terminal half-life” of an antibody, when used herein refers to the time taken for the serum concentration of the polypeptide to be reduced by 50%, in vivo in the final phase of elimination.

In some embodiments, the half-life extension domain is an Fc region. In some embodiments, the Fc region is a heterodimer comprising two Fc polypeptides, wherein the first antigen-binding region is fused to the first Fc polypeptide and the second antigen-binding region is fused to the second Fc polypeptide and wherein the first and second Fc polypeptides comprise asymmetric amino acid mutations that favor the formation of heterodimers over the formation of homodimers. (see e.g. Ridgway et al. (1996) ‘Knobs-into-holes’ engineering of antibody CH3 domains for heavy chain heterodimerization. Protein Eng 9:617). In some embodiments, the CH3 regions of the Fc polypeptides comprise said asymmetric amino acid mutations, preferably the first Fc polypeptide comprises a T366W substitution and the second Fc polypeptide comprises T366S, L368A and Y407V substitutions, or vice versa, wherein the amino acid positions correspond to human IgG1 according to the EU numbering system. In some embodiments, the cysteine residues at position 220 in the first and second Fc polypeptides have been deleted or substituted, wherein the amino acid position corresponds to human IgG1 according to the EU numbering system. In some embodiments, the region comprises the hinge sequence set forth in SEQ ID NO:10.

In some embodiments, the first and/or second Fc polypeptides contain mutations that render the antibody inert, i.e. unable to, or having reduced ability to, mediate effector functions. In some embodiments, the inert Fc region is in addition not able to bind C1q. In some embodiments, the first and second Fc polypeptides comprise a mutation at position 234 and/or 235, preferably the first and second Fc polypeptide comprise an L234F and an L235E substitution, wherein the amino acid positions correspond to human IgG1 according to the EU numbering system. In some embodiments, the antibody contains a L234A mutation, a L235A mutation and a P329G mutation. In some embodiments, the antibody contains a L234F mutation, a L235E mutation and a D265A mutation.

Exemplary bispecific antibodies for use according to the methods described herein are provided in WO 2022/122973, which is incorporated herein by reference.

“Treatment” or “treating” refers to the administration of an effective amount of a bispecific antibody or pharmaceutical composition comprising the same with the purpose of easing, ameliorating, arresting, eradicating (curing) or preventing symptoms or disease states. An “effective amount” refers to an amount effective, at dosages and for periods of time necessary, to achieve a desired therapeutic result. An effective amount of a polypeptide, such as an antibody, may vary according to factors such as the disease stage, age, sex, and weight of the individual, and the ability of the antibody to elicit a desired response in the individual. An effective amount is also one in which any toxic or detrimental effects of the antibody are outweighed by the therapeutically beneficial effects. Administration may be carried out by any suitable route, but will typically be parenteral, such as intravenous, intramuscular or subcutaneous.

In some embodiments, the cancer is metastatic. In some embodiments, the cancer is unresectable (i.e., cannot be removed surgically). In some embodiments, the subject has received prior treatment with one or more chemotherapeutics agents and/or one or more immune checkpoint inhibitors. In some embodiments, the cancer is relapsed after prior treatment with one or more chemotherapeutic agents and/or immune checkpoint inhibitors. In some embodiments, the cancer is refractory to treatment with one or more chemotherapeutic agents and/or immune checkpoint inhibitors.

In some embodiments, the one or more chemotherapeutic agents are selected from a platinum-based chemotherapeutic, a fluoropyrimidine, and a topoisomerase inhibitor. In some embodiments, the platinum-based chemotherapeutic is oxaliplatin. In some embodiments, the topoisomerase inhibitor is irinotecan or camptothecin.

In some embodiments, the one or more chemotherapeutic agents is gemcitabine. In some embodiments, the one or more chemotherapeutic agents comprises leucovorin calcium (folinic acid), fluorouracil, irinotecan hydrochloride, and oxaliplatin (e.g., a FOLFIRINOX regimen).

In some embodiments, the immune checkpoint inhibitor inhibits an immune checkpoint selected from PD1, PDL1, CTLA4, TIM-3, and LAG-3. In some embodiments, the immune checkpoint inhibitor is an anti-PD1 or anti-PDL1 antibody. In some embodiments, the anti-PDL1 antibody is selected from atezolizumab, avelumab, and durvalumab. In some embodiments, the anti-PD1 antibody is selected from nivolumab and pembrolizumab.

In some embodiments, one or more exclusion criterion is applied to select the subject for treatment with the bispecific antibodies described herein. In some embodiments, the one or more exclusion criterion is selected from:
(a) the subject has not been diagnosed with another malignancy less than 3 years prior to administration of a first dose of the bispecific antibody;
(b) the subject does not have an active central nervous system metastases or leptomeingeal disease;
(c) the subject has not received treatment with an aminobisphosphonate IV (e.g., ibandronate, pamidronate, zoledronate) less than 4 weeks prior to administration of a first dose of the bispecific antibody;
(d) the subject has not had a thromboembolic phenomenon (e.g., pulmonary embolism, deep vein thrombosis, stroke, or ischemic attack) less than 6 months prior to administration of a first dose of the bispecific antibody;
(e) the subject is not receiving an anti-coagulation therapy;
(f) the subject is not contraindicated for thromboembolism prophylactic treatment.

In some embodiments, the bispecific antibody used according to the present methods is formulated as a pharmaceutical composition. A pharmaceutical composition described herein may comprise diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition. Further pharmaceutically-acceptable excipients include any and all suitable solvents, dispersion media, coatings, antibacterial and antifungal agents, isotonicity agents, antioxidants and absorption-delaying agents and the like that are physiologically compatible with the bispecific antibody.

In some embodiments, the pharmaceutical composition comprises the bispecific antibody and (a) 5-20 mM of histidine, wherein the composition has a pH between 5.5 and 6.5, or 5-20 mM of sodium acetate, wherein the composition has a pH between 5.0 and 6.0, (b) 250-350 mM sucrose; (c) 0.01%-0.05% (w/v) polysorbate 80; and (d) 0-20 mM methionine. In some embodiments, the pharmaceutical formulation comprises (a) 1 mg/ml or 10 mg/ml of the bispecific antibody; (b) 10 mM Histidine, (c) 280 mM Sucrose, (d) 0.02% Polysorbate 80, (e) pH 6.0, and (f) 1 mM Methionine.

TABLE 1
Sequences

| SEQ ID. | code | Description | Sequence |
|---|---|---|---|
| 22 | 5C8 | CDR1 | NYAMG |
| 2 | 5C8 | CDR2 | AISWSGGSTSYADSVKG |
| 15 | 5C8 | CDR3 | QFSGADYGFGRLGIRGYEYDY |
| 13 | 5C8 | VHH | EVQLVESGGGLVQAGGSLRLSCAASGRPFSNYAMGWFRQAPGKE |
| | | | REFVAAISWSGGSTSYADSVKGRFTISRDNAKNTVYLQMNSPKP |
| | | | EDTAIYYCAAQFSGADYGFGRLGIRGYEYDYWGQGTQVTVSS |
| 1 | 5C8 var | CDR1 | NYAMX₁, wherein X₁ is S or G |
| 21 | 5C8 var1 | CDR1 | NYAMS |
| 2 | 5C8 var | CDR2 | AISWSGGSTSYADSVKG |
| 3 | 5C8 var | CDR3 | QFSGADX₂GFGRLGIRGYEYDY, wherein X₂ is F or S |
| | (Y105) | | |
| 19 | 5C8 var | CDR3 | QFSGADFGFGRLGIRGYEYDY |
| | (Y105F) | | |
| 20 | 508 var | CDR3 | QFSGADSGFGRLGIRGYEYDY |
| | (Y105S) | | |
| 4 | 5C8 var | VHH | EVQLLESGGGSVQPGGSLRLSCAASGRPFSNYAMX₁WFRQAPGKE |
| | | | REFVSAISWSGGSTSYADSVKGRFTISRDNSKNTLYLQMNSLRA |
| | | | EDTAVYYCAAQFSGADX₂GFGRLGIRGYEYDYWGQGTQVTVSS, |
| | | | wherein X₁ is S or G, and wherein X₂ is F or |
| | | | S |
| 14 | 5C8var1 | VHH | EVQLLESGGGSVQPGGSLRLSCAASGRPFSNYAMSWFRQAPGKE |
| | | | REFVSAISWSGGSTSYADSVKGRFTISRDNSKNTLYLQMNSLRA |
| | | | EDTAVYYCAAQFSGADYGFGRLGIRGYEYDYWGQGTQVTVSS |
| 23 | 5C8 var1 | VHH | EVQLLESGGGSVQPGGSLRLSCAASGRPFSNYAMSWFRQAPGKE |
| | (Y105F) | | REFVSAISWSGGSTSYADSVKGRFTISRDNSKNTLYLQMNSLRA |
| | | | EDTAVYYCAAQFSGADFGFGRLGIRGYEYDYWGQGTQVTVSS |
| 24 | 5C8 var1 | VHH | EVQLLESGGGSVQPGGSLRLSCAASGRPFSNYAMSWFRQAPGKE |
| | (Y105S) | | REFVSAISWSGGSTSYADSVKGRFTISRDNSKNTLYLQMNSLRA |
| | | | EDTAVYYCAAQFSGADSGFGRLGIRGYEYDYWGQGTQVTVSS |
| 5 | 7D12 | CDR1 | SYGMG |
| | (EGFR) | | |
| 6 | 7D12 | CDR2 | GISWRGDSTGYADSVKG |
| | (EGFR) | | |
| 7 | 7D12 | CDR3 | AAGSAWYGTLYEYDY |
| | (EGFR) | | |
| 8 | 7D12var8 | VHH | EVQLVESGGGSVQPGGSLRLSCAASGRTSRSYGMGWFRQAPGKE |
| | (EGFR) | | REFVSGISWRGDSTGYADSVKGRFTISRDNAKNTVDLQMNSLRA |
| | | | EDTAVYYCAAAAGSAWYGTLYEYDYWGQGTLVTVSS |
| 9 | | linker | GGGGS |
| 10 | | Modified | AAASDKTHTCPPCP |
| | | hinge | |
| 11 | IgG1 | Heavy chain | AAASDKTHTCPPCPAPEFEGGPSVFLFPPKPKDTLMISRTPEVT |
| | L234F | constant | CVVVDVSHEDPEVKFNWYVDGVEVHNAKTKPREEQYNSTYRVVS |
| | L235E | region | VLTVLHQDWLNGKEYKCKVSNKALPAPIEKTISKAKGQPREPQV |
| | T366W | variant | YTLPPSRDELTKNQVSLWCLVKGFYPSDIAVEWESNGQPENNYK |
| | | | TTPPVLDSDGSFFLYSKLTVDKSRWQQGNVFSCSVMHEALHNHY |
| | | | TQKSLSLSPGX₃, wherein X₃ is K or is absent |
| 12 | IgG1 | Heavy chain | AAASDKTHTCPPCPAPEFEGGPSVFLFPPKPKDTLMISRTPEVT |
| | L234F | constant | CVVVDVSHEDPEVKFNWYVDGVEVHNAKTKPREEQYNSTYRVVS |
| | L235E | region | VLTVLHQDWLNGKEYKCKVSNKALPAPIEKTISKAKGQPREPQV |
| | T366S | variant | YTLPPSRDELTKNQVSLSCAVKGFYPSDIAVEWESNGQPENNYK |
| | L368A | | TTPPVLDSDGSFFLVSKLTVDKSRWQQGNVFSCSVMHEALHNHY |
| | Y407V | | TQKSLSLSPGX₃, wherein X₃ is K or is absent |
| 16 | 7D12var8- | VHH-Fc | EVQLVESGGGSVQPGGSLRLSCAASGRTSRSYGMGWFRQAPGKE |
| | Fc | | REFVSGISWRGDSTGYADSVKGRFTISRDNAKNTVDLQMNSLRA |
| | | | EDTAVYYCAAAAGSAWYGTLYEYDYWGQGTLVTVSSAAASDKTH |
| | | | TCPPCPAPEFEGGPSVFLFPPKPKDTLMISRTPEVTCVVVDVSH |
| | | | EDPEVKFNWYVDGVEVHNAKTKPREEQYNSTYRVVSVLTVLHQD |
| | | | WLNGKEYKCKVSNKALPAPIEKTISKAKGQPREPQVYTLPPSRD |
| | | | ELTKNQVSLSCAVKGFYPSDIAVEWESNGQPENNYKTTPPVLDS |
| | | | DGSFFLVSKLTVDKSRWQQGNVFSCSVMHEALHNHYTQKSLSLS |
| | | | PGX₃, wherein X₃ is K or is absent |
| 17 | 5C8var1 | VHH-Fc | EVQLLESGGGSVQPGGSLRLSCAASGRPFSNYAMSWFRQAPGKE |
| | (Y105F)- | | REFVSAISWSGGSTSYADSVKGRFTISRDNSKNTLYLQMNSLRA |
| | Fc | | EDTAVYYCAAQFSGADFGFGRLGIRGYEYDYWGQGTQVTVSSAA |
| | | | ASDKTHTCPPCPAPEFEGGPSVFLFPPKPKDTLMISRTPEVTCV |
| | | | VVDVSHEDPEVKFNWYVDGVEVHNAKTKPREEQYNSTYRVVSVL |
| | | | TVLHQDWLNGKEYKCKVSNKALPAPIEKTISKAKGQPREPQVYT |
| | | | LPPSRDELTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGX₃, wherein X₃ is K or is absent |
| 18 | 5C8var1 | VHH-Fc | EVQLLESGGGSVQPGGSLRLSCAASGRPFSNYAMSWFRQAPGKE |
| | (Y105S)- | | REFVSAISWSGGSTSYADSVKGRFTISRDNSKNTLYLQMNSLRA |
| | Fc | | EDTAVYYCAAQFSGADSGFGRLGIRGYEYDYWGQGTQVTVSSAA |
| | | | ASDKTHTCPPCPAPEFEGGPSVFLFPPKPKDTLMISRTPEVTCV |
| | | | VVDVSHEDPEVKFNWYVDGVEVHNAKTKPREEQYNSTYRVVSVL |
| | | | TVLHQDWLNGKEYKCKVSNKALPAPIEKTISKAKGQPREPQVYT |
| | | | LPPSRDELTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGX₃, wherein X₃ is K or is absent |

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

1. A method of treating pancreatic cancer in a subject in need thereof comprising administering to the subject a bispecific antibody comprising
a. a first antigen-binding region that binds a Vγ9Vδ2 T cell receptor (TCR) and comprises
i. a CDR1 of SEQ ID NO: 21, a CDR2 of SEQ ID NO: 2, and a CDR3 of SEQ ID NO: 19, or
ii. a CDR1 of SEQ ID NO: 21, a CDR2 of SEQ ID NO: 2, and a CDR3 of SEQ ID NO: 20
b. a second antigen-binding region that binds to EFGR and comprises a CDR1 of SEQ ID NO: 5, a CDR2 of SEQ ID NO: 6, and a CDR3 of SEQ ID NO: 7.

2. A method of treating pancreatic cancer in a subject in need thereof comprising administering to the subject a bispecific antibody comprising
a. a first antigen-binding region that binds a Vγ9Vδ2 T cell receptor (TCR) and comprises an amino acid sequence selected from SEQ ID NO:24 and 23 and
b. a second antigen-binding region that binds to EFGR and comprises an amino acid sequence of SEQ ID NO:8.

3. The method of claim 1 or 2, wherein the pancreatic cancer is metastatic pancreatic cancer

4. The method of any one of claims 1-3, wherein the pancreatic cancer is unresectable.

5. The method of any one of claims 1-4, wherein the pancreatic cancer is pancreatic ductal adenocarcinoma.

6. The method of any one of claims 1-5, wherein the pancreatic cancer is relapsed or refractory pancreatic cancer.

7. The method of any one of claims 1-6, wherein the subject has received prior treatment with one or more chemotherapeutic agents and/or one or more immune checkpoint inhibitors.

8. The method of claim 7, wherein the one or more chemotherapeutic agents are selected from
a. gemcitabine; and
b. leucovorin calcium (folinic acid), fluorouracil, irinotecan hydrochloride, and oxaliplatin.

9. The method of any one of claims 1-8, wherein the subject has not been diagnosed with another malignancy less than 3 years prior to administration of a first dose of the bispecific antibody.

10. The method of any one of claims 1-9, wherein the subject does not have an active central nervous system metastases or leptomeingeal disease.

11. The method of any one of claims 1-10, wherein the subject has not received treatment with an aminobisphosphonate IV less than 4 weeks prior to administration of a first dose of the bispecific antibody.

12. The method of any one of claims 1-11, wherein the subject has not had a thromboembolic phenomena less than 6 months prior to administration of a first dose of the bispecific antibody.

13. The method of any one of claims 1-12, wherein the bispecific antibody is formulated in a pharmaceutical composition comprising:
a. 1 mg/mL or 10 mg/mL of the bispecific antibody;
b. 10 mM Histidine,
c. 280 mM Sucrose,
d. 0.02% Polysorbate 80,
e. pH 6.0, and
f. 1 mM Methionine.

14. The method of any one of claims 1-13, wherein the subject is a human.

15. The method of any one of claims 1-14, wherein the bispecific antibody or pharmaceutical composition comprising the same is administered intravenously.
